(19) United States
(12) Patent Application Publication
(10) Pub. No.: US 2012/0245547 A1
(43) Pub. Date: Sep. 27, 2012

(54) INCONTINENCE GARMENT

(76) Inventors: Nolan M. Wilcox, Columbus, OH (US); Heather J. Wilcox, Columbus, OH (US)

(21) Appl. No.: 13/070,985

(22) Filed: Mar. 24, 2011

(51) Int. Cl. A61F 13/45

(52) U.S. Cl. 604/385.01

(57) ABSTRACT

An incontinence garment is provided which includes a front portion, the front portion including a body, a body absorbent, and a body liquid-permeable liner. The body comprises a shell, which may be flexible and semi-rigid, and which is shaped to form a cavity adapted to hold male genitalia. The body absorbent, suitable for absorbing urine, is at least partially disposed within the cavity and the body liquid-permeable liner is attached to the body in a spaced-apart relation to the body absorbent. The body liquid-permeable liner and the body absorbent describe a gap and at least partially form a chamber. Even while the garment is being worn, the body liquid-permeable liner is under sufficient tension to at least partially maintain the gap and at least partially maintain the at least partial chamber and to hammock male genitalia within the cavity and away from the body absorbent.

### TECHNICAL FIELD

This description relates to absorbent garments, particularly to incontinence garments, and more particularly to incontinence garments for absorbing urine discharge resulting from male urinary incontinence.

### BACKGROUND

Male urinary incontinence may be caused by a variety of factors. It can occur, in varying degrees, in infant males not yet toilet trained, young boys suffering from nocturnal enuresis (bed wetting), men who have undergone a prostatectomy (surgical removal of the prostate), other urinary tract surgeries, such as a cystectomy (surgical removal of the bladder), and prostate radiation, in diabetics with nerve damage, and in men who have had a stroke or spinal cord injury or who suffer from Parkinson's disease or multiple sclerosis. Aging is also a leading cause of male incontinence. Other causes include loss of control from such actions as coughing, sneezing, and lifting, and an abnormal and overwhelming urge to urinate caused, for example, by various bladder conditions such as exposure to radiation therapy. Finally, men who are handicapped or otherwise medically compromised may have problems with incontinence. Urinary incontinence can be more than an inconvenience and an embarrassment to men, it can cause discomfort from being in wet or damp clothing as well as genital itching or a rash or infection caused by contact with discharged urine.

Some conventional absorbent devices to help manage male urinary incontinence include those which are positioned beneath undergarments and next to the genitals. Discharged urine is retained within an absorbent layer while a moisture barrier layer is interposed between the retained urine and the undergarments and other clothing. In this way, discharged urine is retained and not allowed to leak onto, or otherwise soil, clothing. In addition, the anxiety and potential embarrassment of being seen with soiled clothing is avoided. The used absorbent device is then later disposed of at an appropriate time. Such devices include diapers, which enclose the genitals, perineum, and buttocks, and pads, which enclose only the genital region.

While such absorbent devices may be effective in absorbing urine and preventing soiled clothing, a bulky or protruding appearance may call attention to the problem, the genitals may be allowed to come into contact with discharged urine and may become too warm, with resultant discomfort and the chance of rash or infection, and, the device may cause further discomfort by not remaining in place while allowing the wearer to move about freely and engage in normal daily activities.

Thus, there is a need for a male urinary incontinence device that is not only effective, but that fits the male genital anatomy comfortably, allows sufficient freedom of movement to permit normal daily activities, may be worn discretely, and that helps the genitals from becoming too warm and helps prevent the genitals from coming into contact with discharged urine.

Even with regard to a conventional diaper-type garment, however, there is a need for a urinary incontinence device that fits the male genital anatomy comfortably and that helps the genitals from becoming too warm and helps prevent the genitals from coming into contact with discharged urine.

### SUMMARY

Devices of the present invention help prevent soiling of clothing with discharged urine, provide comfort to the male genitalia, allow sufficient freedom of movement to permit normal daily activities, and help separate the genitalia from discharged urine, thus providing a more natural feel. In one embodiment, a male urinary incontinence device includes a body which has a shell which forms a cavity adapted to hold male genitalia. Disposed within the cavity is an absorbent suitable for absorbing and storing discharged urine. Alternatively, the shell may be at least partially absorbent, thus obviating the need for a separate absorbent. A moisture barrier may be placed adjacent the body and placed between the absorbent and the clothing, or outboard, of the wearer to help prevent soiling of the clothing. A liquid-permeable liner, such as a mesh-like material, for example, is attached to the body so as to provide a gap between the liner and the absorbent. This hammock-like effect creates an air space, or an at least partial chamber, and holds the male genitalia away from the discharged urine stored in the absorbent. When necessary, the device is removed and discarded.

In a further embodiment, the shell is formed from a flexible, semi-rigid shell. The body may further include a flexible, non-rigid crest extending from the shell at the top of the device and may further include a flexible, non-rigid tongue extending from the shell at the bottom of the device. In a further embodiment, the crest extends between about 15 percent and about 20 percent of the length of the body. In a further embodiment, the tongue extends between about 25 percent and about 30 percent of the length of the body. In a further embodiment, the tongue forms an angle of between about 30 degrees and about 50 degrees.

In a further embodiment, the device forms a length external angle of between about 70 degrees and about 100 degrees. In a further embodiment, the device forms a width internal angle of between about 85 degrees and about 105 degrees.

In a further embodiment, an incontinence garment comprises a front portion which includes a body comprising a shell and the shell forms a cavity which is shaped to at least partially accommodate male genitalia. Further included is a body absorbent at least partially disposed within the cavity of the shell. The body absorbent is preferably designed to absorb and hold urine. Attached to the shell is a body liquid-permeable liner which is spaced apart from the body absorbent so that the body liquid-permeable liner and the body absorbent describe a gap and form at least a partial chamber. The incontinence garment further comprises a rear portion which is operatively connected to the front portion.

In a further embodiment, the incontinence garment includes an outer moister barrier which moisture barrier is positioned outboard of, or away from the wearer of, of the body absorbent.

In a further embodiment, the shell has a perimeter with a shape and a length and the body liquid-permeable liner also has a perimeter with a shape and a length. The perimeter of the liquid-permeable liner is attached to the perimeter of the shell. In a further embodiment, the length and shape of the liquid-permeable liner perimeter and the length and shape of the shell perimeter are approximately equal. In a further embodiment, when the incontinence garment is being worn, the liquid-permeable liner is under sufficient tension to at least partially maintain the gap and the at least partial chamber and to hammock male genitalia within the cavity and away from the body absorbent.

In a further embodiment, the shell is flexible and semi-rigid.

In a further embodiment, the body further comprises a flexible, non-rigid crest, the crest extending toward the navel of the wearer upward from the shell.

In a further embodiment, the body further comprises a flexible, non-rigid tongue, the tongue extending from the shell, and wherein, the tongue extends toward the perineum of the wearer downward from the shell.

In a further embodiment, the front portion further comprises a front portion absorbent, the front portion absorbent in a spaced-apart relation to the body absorbent.

In a further embodiment, a first front portion moisture barrier is interposed between the body absorbent and the front portion absorbent.

In a further embodiment, the front portion further comprises a front portion liquid-permeable liner positioned inboard of the front portion absorbent, the front portion liquid-permeable liner in a spaced-apart relation to the body liquid-permeable liner.

In a further embodiment, a second front portion moisture barrier is interposed between the body liquid-permeable liner and the front portion liquid-permeable liner.

In a further embodiment, an incontinence garment comprises a chassis, the chassis having an interior and an exterior. The chassis further comprises a chassis moisture barrier, a chassis absorbent positioned inboard of the chassis moisture barrier, a chassis liquid-permeable liner positioned inboard of the chassis absorbent, and an aperture, the aperture having a perimeter, and the aperture defining an opening between the chassis interior and at least the chassis moisture barrier.

The incontinence garment further comprises a body, the body having a perimeter, the body perimeter positioned adjacent the aperture perimeter. The body comprises a shell, the shell forms a cavity, the cavity adapted to hold male genitalia. The body further comprises a body absorbent, the body absorbent at least partially disposed within the cavity. The body further comprises a body liquid-permeable liner in a spaced-apart relation to the body absorbent, the body liquid-permeable liner and the body absorbent describing a gap and forming at least a partial chamber. While the incontinence garment is being worn, the body liquid-permeable liner is under sufficient tension to at least partially maintain the gap and to at least partially maintain the at least partial chamber and to hammock the male genitalia within the cavity and away from the body absorbent.

In a further embodiment, the body forms a length external angle of between about 70 degrees and about 100 degrees.

In a further embodiment, the body forms a width internal angle of between about 85 degrees and about 105 degrees.

In a further embodiment, the ratio of a width of the body along a short axis to a length of the body along a long axis is between about 45 percent and about 65 percent.

In a further embodiment, the incontinence garment includes a first incontinence garment moisture barrier, the first incontinence garment moisture barrier interposed between the body absorbent and the chassis absorbent.

In a further embodiment, the incontinence garment includes a second incontinence garment moisture barrier, the second incontinence garment moisture barrier interposed between the body liquid-permeable liner and the chassis liquid-permeable liner.

Additional objects, features, and advantages of the invention will become apparent to those skilled in the relevant art upon consideration of the following detailed description of preferred embodiments exemplifying the best mode of carrying out the invention as presently perceived.

### BRIEF DESCRIPTION OF THE SEVERAL VIEWS OF THE DRAWING

The accompanying drawings, which are incorporated in, and constitute a part of, this specification, illustrate several embodiments consistent with the invention and, together with the description, serve to explain the principles of the invention. For purposes of illustration, drawings may not be to scale.

FIG. 1 is a perspective view of a male urinary incontinence device according to an embodiment of the present invention.

FIG. 2 is a cross-sectional view taken generally along the plane of the line 2-2 in FIG. 1 and illustrating an embodiment of the present invention.

FIG. 3 is a cross-sectional view taken generally along the plane of the line 3-3 in FIG. 1 and illustrating an embodiment of the present invention.

FIG. 4 is a view of an interior side of a male urinary incontinence device according to an embodiment of the present invention.

FIG. 5 is a cross-sectional side view of an embodiment of the present invention illustrated in FIGS. 1 and 2 and including a representation of the male anatomy while the device is in use.

FIG. 6 is a view of an interior side of a male urinary incontinence device according to an embodiment of the present invention.

FIG. 7 is a cross-sectional view taken generally along the plane of the line 7-7 in FIG. 6 and illustrating an embodiment of the present invention.

FIG. 8 is a top elevation view of a male urinary incontinence device according to an embodiment of the present invention.

FIG. 9 is a right side elevation view of an incontinence garment according to an embodiment of the present invention.

FIG. 10 is an isometric view of the bottom front of an incontinence garment according to an embodiment of the present invention.

FIG. 11 is a partial cross-sectional view taken along the plane of the line 11-11 in FIG. 10.

FIG. 12 is a view according to the “FIG. 12” arrow shown in FIG. 9.

FIG. 13 is a further partial cross-sectional view taken along the plane of the line 13-13 in FIG. 10.

### DETAILED DESCRIPTION

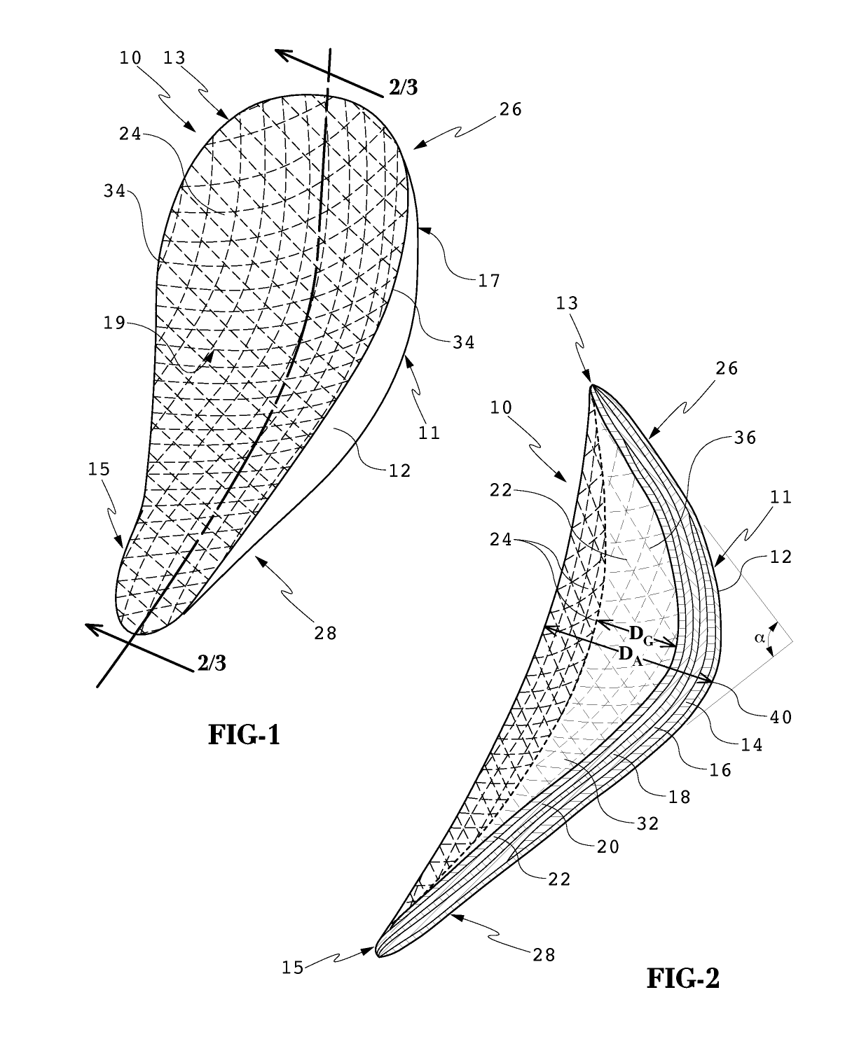

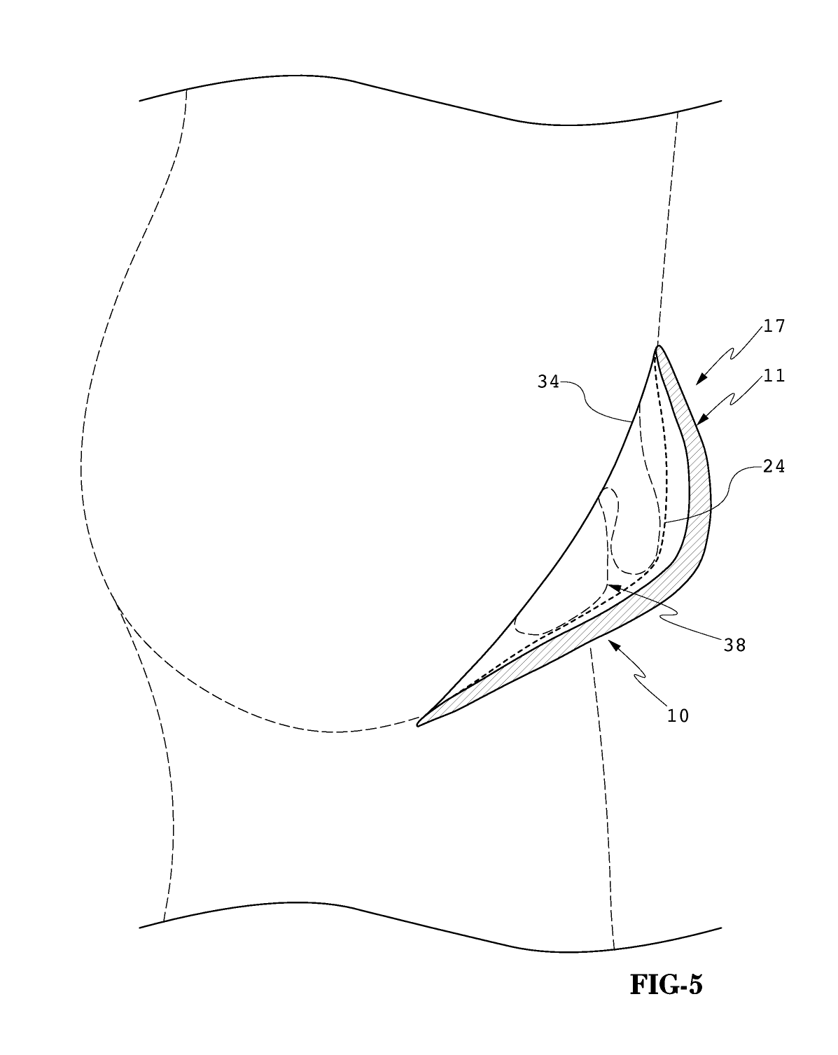

In one embodiment, shown in FIG. 1, a male incontinence device 10 includes a body 11 and a first liquid-permeable liner 24. The device 10 has a top 13 which, when worn, points generally toward the navel of the wearer, and a bottom 15 which, when worn, points generally toward the perineum of the wearer. The device 10 further has an external side 17 which, when worn, faces away from the wearer, and an interior side 19 which, when worn, faces the wearer and, particularly, the genitalia 38 of the wearer. See, FIG. 5.

The body 11 forms a cavity 36 (see, also, FIGS. 2, 3, 5, and 7) adapted to hold human male genitalia 38 and includes a shell 14 and may further include a crest 26 at the top 13 and may further include a tongue 28 at the bottom 15. Note that the neither the crest 26 nor the tongue 28 are shown in FIGS. 5 and 7. Especially when the shell 14 is comprised of flexible, semi-rigid material as described below, the device 10 is easily moved to the side while being worn to facilitate normal male urination. The device 10 also includes at least one moisture barrier 12, 16 adapted to prevent moisture, and specifically urine, from wetting the clothes of the wearer.

As best seen in FIGS. 2, 3, and 7, but also in FIG. 5, the first liquid-permeable liner 24 cooperates with the body 11, and particularly the shell 14 to create a gap D_{G} and a chamber 32. This effect is aided by securing the first liquid-permeable liner 24, having an area sufficient to prevent its total collapse into the body 11. Thus, the first liquid-permeable liner 24 is sufficiently suspended to create the gap D_{G} and the chamber 32 and acts to hold the male genitalia 38 separated from discharged urine stored in one or more absorbent 18, 20. In this configuration, the first liquid-permeable liner 24 also provides needed support to the male genitalia 38. The gap D_{G} and the chamber 32 also create an opportunity for air to circulate about the genitals 38 for a further drying effect. While the first liquid-permeable liner 24 may be secured about the periphery 34 of the body 11, the first liquid-permeable liner 24 may also be secured inboard of the periphery 34.

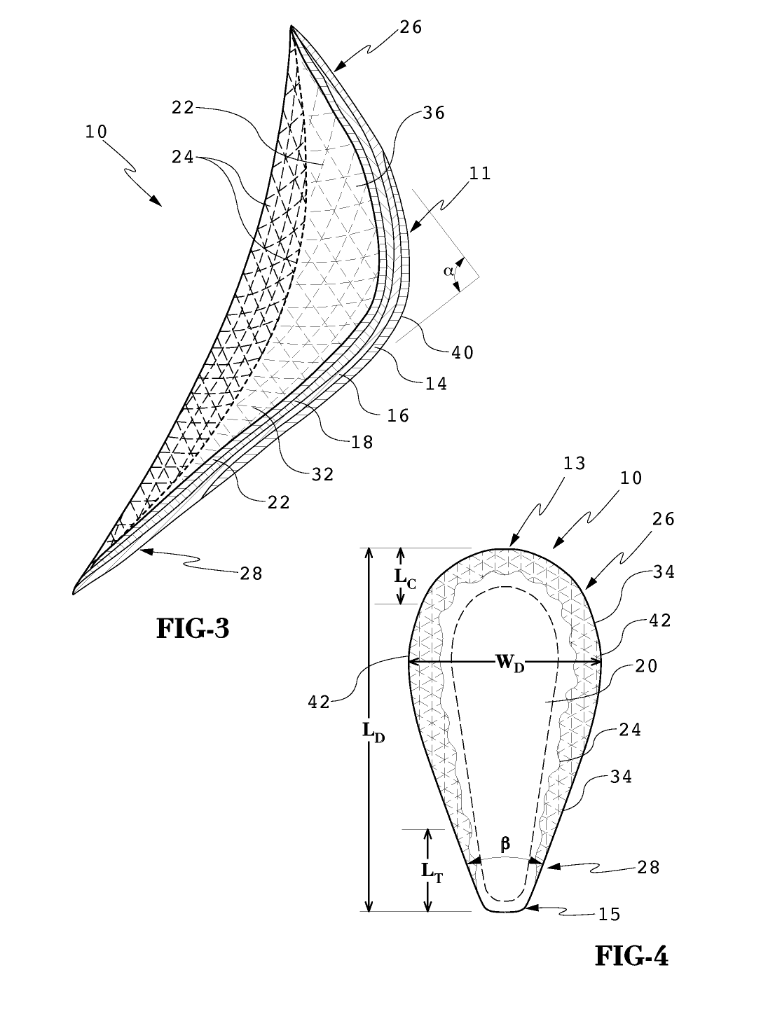

As shown in FIGS. 2 and 3, the device 10 forms a length external angle α and, as shown in FIG. 8, a width internal angle θ. The length external angle α is that angle formed by the intersection of extending a tangent from the top 13 of the device 10 and from the bottom 15 of the device 10. The width internal angle θ is that angle formed between an apex 40 of the external side 17 of the device 10 and edges 42 at the widest point W_{D} of the device 10.

The length external angle a cooperates with the width external angle θ to accommodate human male genitalia 38 (FIG. 5) and to minimize the characteristic visible “bulge” indicative of men wearing an incontinence device. The length external angle α is between about 70 deg. and about 100 deg. The width internal angle θ is between about 85 deg. and about 105 deg. Proportionally, the width internal angle θ is between about 85 percent and 150 percent of the length external angle α.

As shown in FIGS. 2 and 3, the crest 26 extends from the shell 14 and generally comprises soft, flexible material. The crest 26 forms a comfortable seal at the abdomen of the wearer and can provide additional absorbent protection. Also shown in FIGS. 2 and 3, the tongue 28 extends from the shell 14 and also generally comprises soft, flexible material. The tongue 28 forms a comfortable seal beneath the scrotum and in the perineum. Additionally, the tongue 28 may provide further absorbent protection. The tongue 28 forms an angle β of between about 30 deg. and 50 deg. Both the crest 26 and the tongue 28 provide a comfortable transition from the device 10 to the human body.

In an embodiment, the periphery 34 of the device 10 comprises soft, flexible material for comfort and to help seal the device 10 against the body of the wearer.

As shown in FIGS. 2-4 and in Table 1, below, the device 10 has an overall length L_{D}, a width at the widest part W_{D}, a depth at the apex 40 of D_{A}, a tongue length L_{T}, and a crest length L_{C}. Proportionally, the width W_{D} is between about 50 percent and about 55 percent of the overall length L_{D}. As will be appreciated by those skilled in the relevant art, the indicated sizes are exemplary only, and smaller, larger, and other intermediate sizes are possible without departing from the spirit of the invention.

Table 1, below, lists representative dimensions for four different sized devices 10. All dimensions are in centimeters (cm).

TABLE 1

| Size | L_{D} | W_{D} | D_{A} | L_{T} | L_{C} |
|---|---|---|---|---|---|
| Small (cm) | 16.5 | 9 | 5 | 5 | 3.5 |
| Medium (cm) | 17.5 | 9.5 | 5.5 | 5 | 3.5 |
| Large (cm) | 19 | 9.5 | 6.0 | 5 | 3.5 |
| Extra-Large (cm) | 19.5 | 10 | 6.0 | 5 | 3.5 |

In one embodiment, the shell 14 is flexible and semi-rigid and may be molded from a sheet or batt of fibers by placing the fiber sheet or batt between dies and, with the application of heat and pressure, molded into the desired shape. The shell 14 may also be resilient and attempt to return to its original shape when distorting forces are removed. Conventional examples include materials used to make particle masks so manufactured using natural fibers, such as wool and cotton, and synthetic materials, such as nylon, acrylonitrile, polyethylene, polyester, or polypropylene fibers. These fibers are often mixed with chemical resins, such as polyolefinic resins The nominal thickness of the shell 14 is about 2 mm. As will be appreciated by those skilled in the relevant art, various foams and other plastics may also be employed in making the shell 14. The shell 14 is flexible with the pressures due to normal activities while being worn. At the same time, the shell 14 is resistant to flattening out against the genitalia 38. The latter may be aided in part, for example, by molding into the shell 14, elongated ridges to improve shape retention as may be seen in conventional dust masks. An exemplary shell 14 that has been used successfully uses the material of the 8000 series N95 Particle Mask from 3M, St. Paul, Minn. Other, non-limiting, examples include those found in U.S. Pat. No. 4,384,577 to Huber et al. and U.S. Pat. No. 3,220,409 to Liloia et al.

In a further embodiment, the shell 14 comprises rigid material, plastic, for example. In such an embodiment, the shell 14 can provide further strength to help keep the body 11 from collapsing around the genitalia 38, in the case of penile, testicular, or scrotal surgery, for example.

The first liquid-permeable liner 24 is in contact with the genitalia 38 and serves to allow discharged urine to pass through freely and to keep the genitalia separated from the discharged urine. Preferably, the first liquid-permeable liner 24 is compliant, soft to the touch, and non-irritating. In addition, the first liquid-permeable liner 24 is non-absorbent and preferably formed from rapidly drying material which wicks away moisture quickly. Typically, the liner 24 is a netting material, woven or non-woven, comprised of, for example, polyester, nylon, polypropylene, rayon, or cotton. Other materials include apertured hydrophobic formed film. Exemplary, but non-limiting, formed films include those described in U.S. Pat. No. 4,324,246 to Mullane et al. and U.S. Pat. No. 4,342,314 to Radel et al. Other, non-limiting, liner materials include that described in U.S. Pat. No. 5,705,249 to Takai et al. In an exemplary embodiment, the first liquid-permeable liner 24 comprises mesh-like material.

A first absorbent 18 provides collection and storage of discharged urine and is formed of material adapted to absorb and retain human urine. Successful materials include processed wood pulp and super-absorbent polymers found in conventional disposable diapers and feminine hygiene products, such as polyacrylate/polyacrylamide copolymers, ethylene maleic anhydride copolymer, cross-lined carboxy-methyl-cellulose, polyvinyl alcohol copolymers, cross-linked polyethylene oxide, and starch grafted copolymer of polyacrylonitrile.

Turning now to FIGS. 2 and 4, a second absorbent 20 may be included to provide additional absorbent capacity. As shown in FIG. 4, the second absorbent may be limited to a central portion of the device 10 where the majority of the discharged urine would collect. Accordingly, the second absorbent 20 may provide a higher absorbent density so that additional urine can be effectively absorbed and stored.

Exemplary absorbent materials that have been used successfully include those of Always® ultra thin™ pads (Procter & Gamble, Cincinnati, Ohio). See, for example, U.S. Pat. No. 4,950,264 to Osborn, III; U.S. Pat. No. 5,520,875 to Wnuk et al.; and U.S. Pat. No. 6,601,705 to Molina et al. For other, non-limiting, absorbent materials, see, also, U.S. Pat. No. 5,075,344 to Johnson; U.S. Pat. No. 3,926,891 to Gross et al.; and U.S. Pat. No. 4,293,609 to Erickson.

In a further exemplary embodiment, the absorbent materials need not be separable elements, but may be at least partially combined with other elements. For example, the shell 14 may be at least partially absorbent.

In a further embodiment, a second liquid-permeable liner 22 may be provided adjacent the absorbent 18, 20. The second liquid-permeable liner 22 may comprise any suitable material for enclosing the absorbent 18, 20. Exemplary materials include apertured hydrophobic formed films discussed above as well as woven and non-woven materials.

Turning again to FIGS. 2, 3, and 7, in a further embodiment, a first moisture barrier 12 is provided on the external side 17 of the device 10. A sheet of thin plastic of the type commonly found in conventional incontinence devices and feminine pads have been used successfully.

In a further embodiment, shown in FIGS. 2 and 3, a second moisture barrier 16 is provided adjacent to the absorbent 18. As with the first moisture barrier 12, the second moisture barrier 16 of the type of thin plastic sheet commonly used in conventional incontinence devices and feminine pads have been used successfully.

In a further exemplary embodiment, the absorbent materials discussed herein may be at least partially combined with the first moisture barrier 12 and the second moisture barrier 16. By way of example only, an absorbent element may comprise a coating that acts as a moisture barrier.

Turning now to FIG. 3, an embodiment of a male urinary incontinence device 10 is shown which includes a shell 14, a moisture barrier 16, an absorbent 18, and a second liquid-permeable liner 22. The embodiment shown in FIG. 3 does not include a moisture barrier 12 on the external side of the device 10 and includes just one absorbent 18. The first liquid-permeable liner 24 provides support for the genitalia 38 and helps separate the genitalia 38 from urine stored in the absorbent 18. The shell 14 provides shape for the body 11 and helps to prevent the device 10 from collapsing around the genitalia 38. Finally, the moisture barrier 16, placed between the absorbent 18 and the clothing of the wearer to help protect the clothing from becoming soiled.

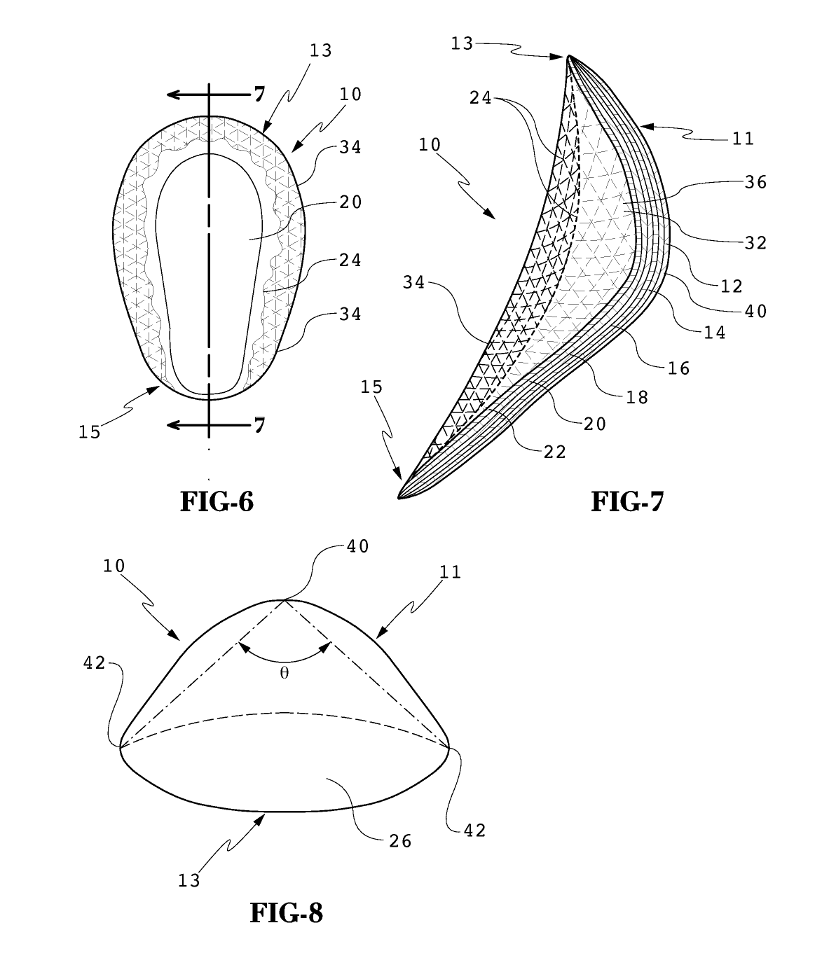

Turning now to FIGS. 6 and 7, an embodiment of a male urinary incontinence device 10 is shown which includes neither a crest 26 nor a tongue 28.

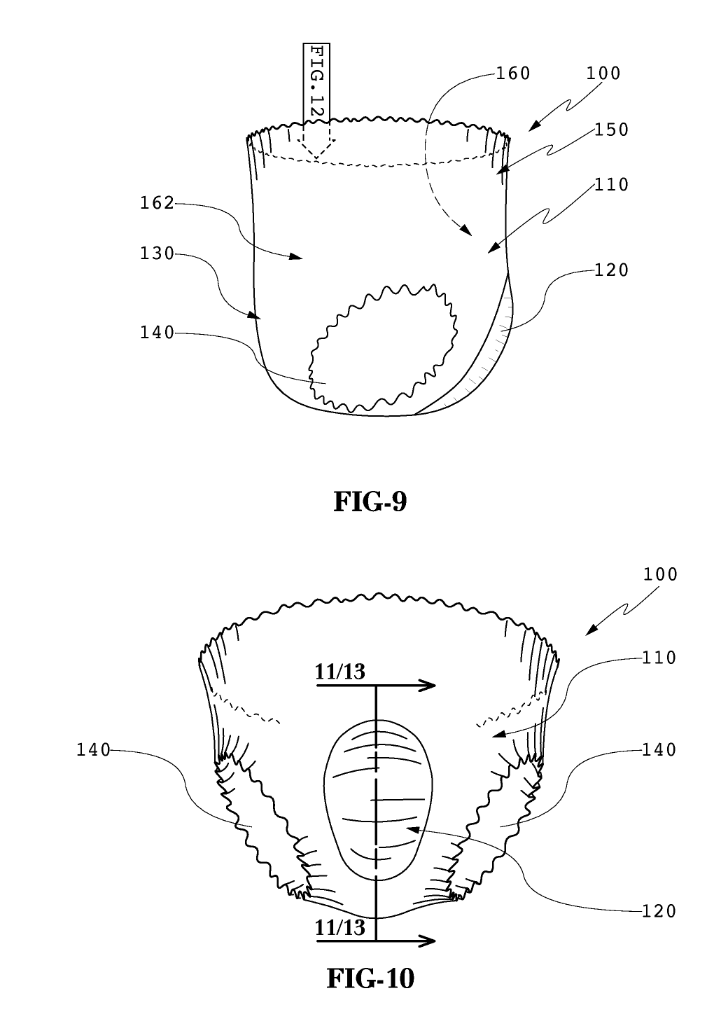

Turning now to FIGS. 9 and 10, an embodiment of an incontinence garment 100 is shown. The garment 100 is generally in the shape of a diaper and is designed to at least partially enclose the lower abdomen of the wearer. Specifically, the garment 100 covers the genitals to absorb discharged urine and may also cover the anus area to collect discharged feces. The garment 100 may be generally a chassis 150 which may include many of the features normally found in a conventional disposable diaper. The chassis 150 includes a front portion 110 and a rear portion 130. The front portion 110 and the rear portion 130 cooperate to enclose the lower abdomen of the wearer. Leg holes or similar openings 140 are provided for a better fit. The garment 100 may be of the pull-up variety, as shown, or open and fit to the wearer with tabs or other closures (not shown). The chassis 150, as shown, includes an interior 160 and an exterior 162. Included as a feature of the garment 100 is a body 120. The features of the body 120 will be further described in more detail herein below. As will be appreciated by those skilled in the relevant art, the body 120 provides many of the features of the male incontinence device 10 described herein above and generally illustrated in FIGS. 1-8. The garment 100, however, integrates those features into standalone wearable article which provides the hammock-like effect for male genitalia as described herein above.

As shown in FIGS. 9 and 10, the body 120 is located at the lower front of the chassis 150 to cooperate with the genitalia of the wearer, particularly male genitalia. (See, also, FIG. 5.)

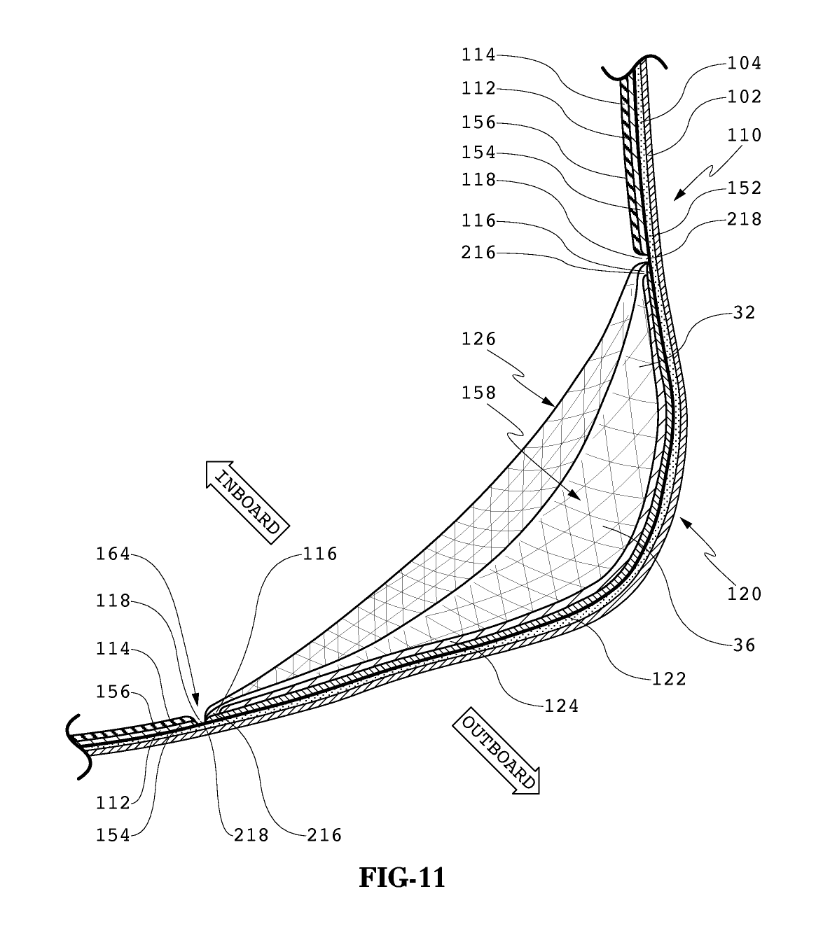

Turning now to FIG. 11, a cross-section view of a portion of the garment 100 is shown which illustrates the detail of the body 120 and its environs. As described generally herein above, the body 120 comprises a shell 122. The shell 122 forms a cavity 36 (see, also, generally, FIGS. 2, 3, and 7) which is adapted to hold male genitalia 38 (FIG. 5). As with the male urinary incontinence device 10, the shell 122 may be comprised of flexible, semi-rigid material, and may be molded from a sheet or batt of fibers by placing the fiber sheet or batt between dies and, with the application of heat and pressure, molded into the desired shape. The shell 122 may also be resilient and attempt to return to its original shape when distorting forces are removed. Further discussion of materials for the shell 122 is found herein above. As will be appreciated by those skilled in the relevant art, numerous conventional materials are available for constructing the shell 122.

The body 120 further comprises a body absorbent 124. As described herein above, there are many materials that are suitable for the body absorbent 124. As shown in FIG. 11, the body absorbent 124 may be placed adjacent to, and conform with, the shell 122. The shell 122 may also be constructed to additionally provide the absorbent effect of the body absorbent 124.

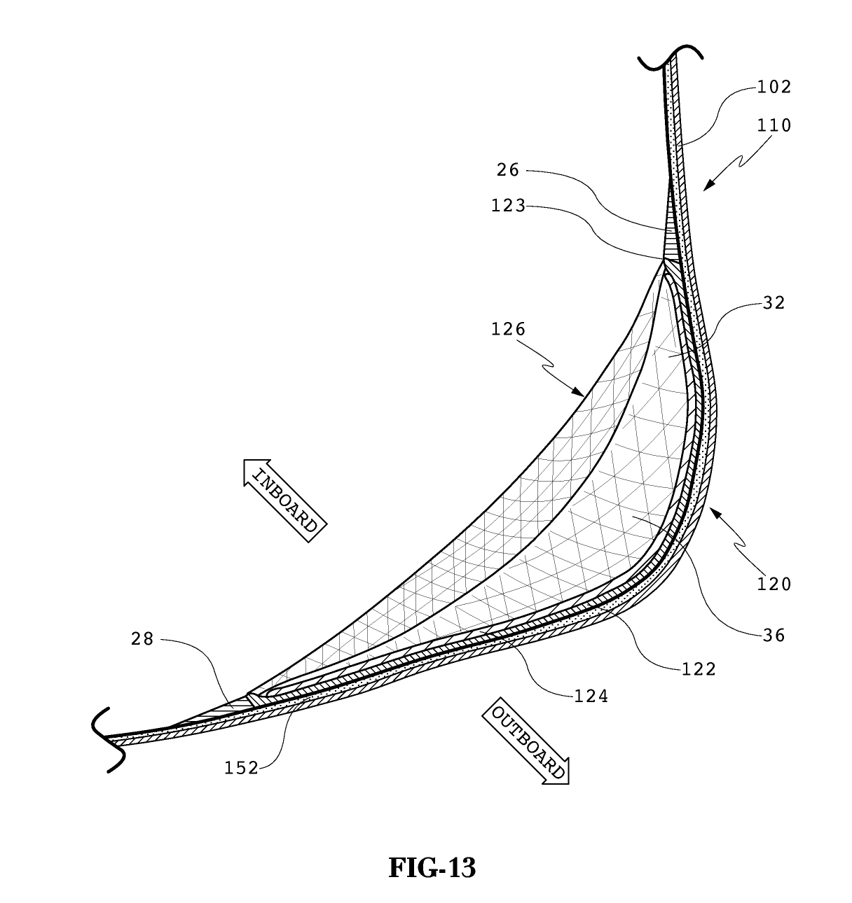

The body 120 further comprises a body liquid-permeable liner 126. Referring back to FIGS. 2, 3, 5, and 7, as well as FIGS. 11 and 13, the body liquid-permeable liner 126 cooperates with the shell 122 to create a gap D_{G} (FIG. 2) and at least a partial chamber 32, which chamber 32 is defined by the body liquid-permeable liner 126 and the body absorbent 124. The body liquid-permeable liner 126 is sized and shaped such that at least a portion of the gap D_{G} (FIG. 2) is maintained even while the garment 110 is being worn. In use, the liquid-permeable liner 126 operates to hold the male genitalia 38 (FIG. 5) separated from discharged urine stored in the body absorbent 124. The body absorbent 124 may only partially cover the inboard surface of the shell 122 or it may completely cover the inboard surface of the shell 122. Thus, the chamber 32 may only be a partial chamber 32, not completely enclosed in the first instance, and a completely enclosed chamber 32 in the second instance.

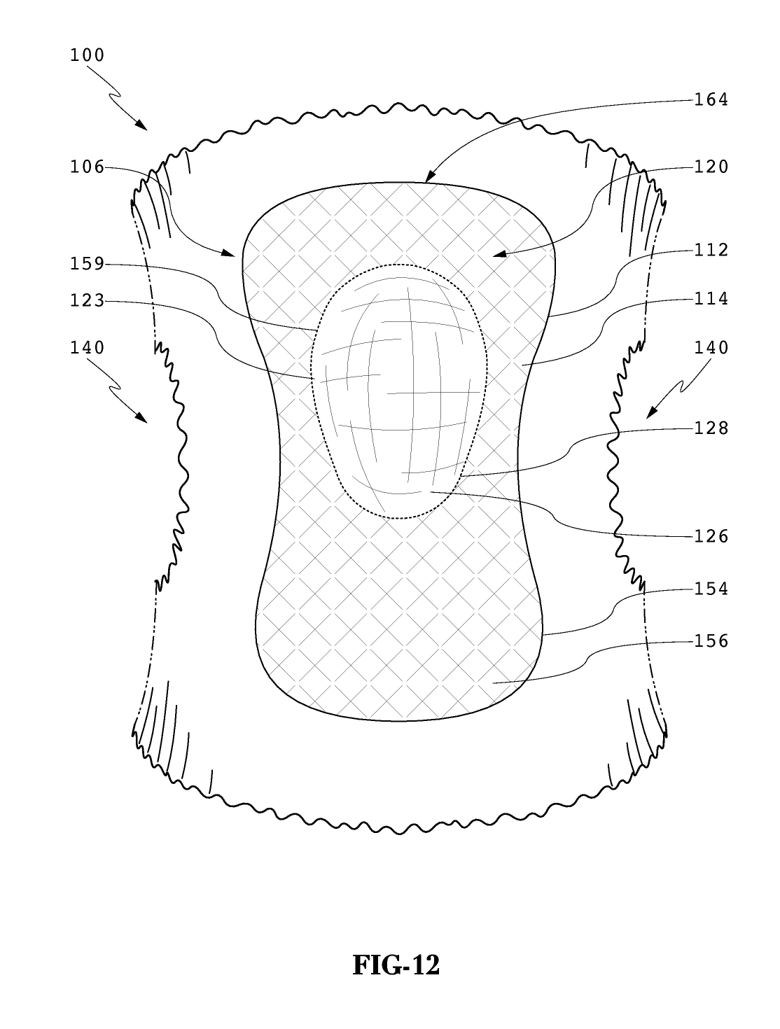

The shell 122 comprises a shell perimeter 123 and the body liquid-permeable liner 126 comprises a body liquid-permeable liner perimeter 128 which cooperate to provide tension on the body liquid-permeable liner 126. See, FIG. 12. It is this tension, maintained and enhanced while the garment 100 is being worn, that provides the hammock-like effect to hold the genitalia 38 of the wearer away from the body absorbent 124. Exemplarily, the shell 122, while constructed so as to be flexible and semi-rigid, possesses sufficient shape integrity to cooperate with the body liquid-permeable liner 126 during use to maintain the genitalia 38 away from the body absorbent 124.

As shown in FIGS. 11 and 13, an outer moisture barrier 104 may be positioned outboard of the body absorbent 124. While shown exemplarily in FIGS. 11 and 13 outboard of the shell 122 also, the purpose of the outer moisture barrier 104 is to keep moisture, such as discharged urine, from migrating to the outside of the garment 100 and thus possible soil the outer clothing of the wearer. (Shown as chassis moisture barrier 152 in FIG. 13.) Also, while FIGS. 11 and 13 show the outer moisture barrier 104/chassis moisture barrier 152 extending into other portions of the garment 100, such as the front portion 110, the outer moisture barrier 104 may be limited to just the body 120 and, particularly, to the body absorbent 124 and the other portions of the garment 100, such as the front portion 110, and even the rear portion 130 (FIG. 9) may include separate means for keeping moisture from migrating to the outside of the garment 100.

Turning now to FIGS. 2-4, 8, and 13, in an exemplary embodiment, the body 120 further comprises a flexible, non-rigid crest 26 which extends upward from the shell 122 toward the navel of the wearer. There also may be provided a flexible, non-rigid tongue 28 which extends downward and slightly backward from the shell 122 toward the perineum of the wearer.

Turning now to FIGS. 11 and 12, in an exemplary embodiment, the front portion 110 comprises a front portion absorbent 112. The front portion absorbent 112 is spaced apart from the body absorbent 124 to help eliminate wicking of liquid between the body absorbent 124 and the front portion absorbent 112. Interposed between the body absorbent 124 and the front portion absorbent 112 may be a first front portion moisture barrier 116 (best seen in FIG. 11). While the first front portion moisture barrier 116 may be a physical separation as shown in FIG. 11, material, such as liquid-impermeable plastic may be employed or the elements bonded such that the body absorbent 124 and the front portion absorbent 112 do not wick from one to the other. In a further exemplary embodiment, the first front portion moisture barrier 116 extends around the shell perimeter 123 and an aperture perimeter 159 or, alternatively, around the body perimeter 164 and the aperture perimeter 159.

In a further exemplary embodiment, as shown in FIGS. 11 and 12, the front portion 110 further comprises a front portion liquid-permeable liner 114. The front portion liquid-permeable liner 114 is spaced apart from the body liquid-permeable liner 126 to help eliminate wicking of liquid between the body liquid-permeable liner 126 and the front portion liquid-permeable liner 114. Interposed between the body liquid-permeable liner 126 and the front portion liquid-permeable liner 114 may be a second front portion moisture barrier 118 (best seen in FIG. 11). While the second front portion moisture barrier 116 may be a physical separation a shown in FIG. 11, material, such as liquid-impermeable plastic may be employed or the elements bonded such that the body liquid-permeable liner 126 and the front portion liquid-permeable liner 114 do not wick from one to the other. In a further exemplary embodiment, the second front portion moisture barrier 118 extends around the shell perimeter 123 and the aperture perimeter 159 or, alternatively, around the body perimeter 164 and the aperture perimeter 159.

Looking next at FIG. 9, in a further exemplary embodiment, the incontinence garment 100 comprises a chassis 150 having a chassis interior 160 and a chassis exterior 162. The chassis 150 further comprises a chassis moisture barrier 152 (FIG. 11), a chassis absorbent 154 (FIGS. 11 and 12) positioned inboard of the chassis moisture barrier 152 (FIG. 11), and a chassis liquid-permeable liner 156 (FIG. 12) positioned inboard of the chassis absorbent 154 (FIG. 11).

Looking next at FIG. 11, an aperture 158, having a perimeter 159 (FIG. 12), defines an opening between the chassis interior 160 and at least the chassis moisture barrier 152. A body 120, having a perimeter 164 (FIGS. 11 and 12), is positioned adjacent the aperture perimeter 159, and is adapted to fit snuggly within the aperture 158. The body 120 comprises a shell 122 and the shell 122 forms a cavity 36. As above, the cavity 36 is adapted to hold male genitalia 38 (FIG. 5). A body absorbent 124 is at least partially disposed within the cavity 36. A body liquid-permeable liner 126 is attached to the shell 122 as shown in FIG. 11 and is sized and under sufficient tension to maintain the body liquid-permeable liner 126 in a spaced-apart relation to the body absorbent 124 and to describe a gap D_{G} (FIG. 2) and form at least a partial chamber 32. The body liquid-permeable liner perimeter 128 is sized to match the shell perimeter 123. In use, while the garment 100 is being worn, the body liquid-permeable liner 126 at least partially maintains the gap D_{G} and at least partially maintains the at least partial chamber 32. Thus, the male genitalia 38 are held as in a hammock by the body liquid-permeable liner 126.

A first incontinence garment moisture barrier 216 is interposed between the body absorbent 124 and the chassis absorbent 154 and a second incontinence garment moisture barrier 218 is interposed between the body liquid-permeable liner 126 and the chassis liquid-permeable liner 156. As described herein above, the first and second garment moisture barriers 216, 218 help prevent wicking of liquid between the respective body absorbent 124 and the chassis absorbent 154 and the body liquid-permeable liner 126 and the chassis liquid-permeable liner 156.

Turning now to FIG. 13, a portion of an incontinence garment 100 (FIGS. 9 and 10) also includes a crest 26 and a tongue 28. Detailed descriptions of the crest 26 and the tongue 28 are included herein above, particularly in association with FIGS. 2 and 3.

As also shown in FIG. 13, in an exemplary embodiment, the body 120 and the body liquid-permeable liner 126 are offset inboard of the front portion 110. Such positioning places the body 120 and the body liquid-permeable liner 126 closer to the wearer relative to the front portion 110 and helps keep the body 120 and the body liquid-permeable liner 126 in contact with the wearer. In general, an inboard offset of about one-quarter inch will be sufficient, although greater offsets may be required.

Experimental Results

In a series of tests, a size small device 10 was used and held in a position similar to that which it would be in when worn and water streamed from a syringe-type delivery apparatus with a slit to mimic the action of urine being excreted from a male penis. In two tests, the absorbent from an Always® Regular feminine hygiene pad absorbed 70 ml before reaching saturation. In one test, the absorbent from an Always® Overnight feminine hygiene pad absorbed 120 ml before reaching saturation.

While certain preferred embodiments of the present invention have been disclosed in detail, it is to be understood that various modifications may be adopted without departing from the spirit of the invention of scope of the following claims.

We claim:

1. An incontinence garment, comprising:
a front portion, the front portion comprising:
a body, the body comprising a shell, the shell forming a cavity, the cavity adapted to at least partially accommodate male genitalia;
a body absorbent, the body absorbent at least partially disposed within the cavity; and
a body liquid-permeable liner, the body liquid-permeable liner attached to the shell, a portion of the body liquid-permeable liner in a spaced-apart relation to the body absorbent, wherein the body liquid-permeable liner and the body absorbent describe a gap and at least partially form a chamber; and
a rear portion, the rear portion operatively connected to the front portion.

2. The incontinence garment of claim 1, further comprising an outer moisture barrier, the outer moisture barrier positioned outboard of the body absorbent.

3. The incontinence garment of claim 1, wherein:
the shell comprises a perimeter, the shell perimeter having a shape and a length;
the body liquid-permeable liner comprises a perimeter, the body liquid-permeable liner perimeter having a shape and a length; and, wherein:
the body liquid-permeable liner perimeter is attached to the shell perimeter.

4. The incontinence garment of claim 3, wherein:
the body liquid-permeable liner perimeter length approximates the shell perimeter length.

5. The incontinence garment of claim 4, wherein:
the shape of the body liquid-permeable liner perimeter approximates the shape of the shell perimeter.

6. The incontinence garment of claim 1, wherein, while the incontinence garment is being worn, the body liquid-permeable liner is under sufficient tension to at least partially maintain the gap and at least partially maintain the at least partial chamber and to hammock male genitalia within the cavity and away from the body absorbent.

7. The incontinence garment of claim 1, wherein the shell is flexible and semi-rigid.

8. The incontinence garment of claim 1, wherein the body further comprises a flexible, non-rigid crest, the crest extending upward from the shell.

9. The incontinence garment of claim 1, wherein the body further comprises a flexible, non-rigid tongue, the tongue extending from the shell, and, wherein the tongue extends downward from the shell.

10. The incontinence garment of claim 1, wherein the front portion further comprises a front portion absorbent, the front portion absorbent in a spaced-apart relation to the body absorbent.

11. The incontinence garment of claim 10, wherein a first front portion moisture barrier is interposed between the body absorbent and the front portion absorbent.

12. The incontinence garment of claim 10, wherein the front portion further comprises a front portion liquid-permeable liner positioned inboard of the front portion absorbent, the front portion liquid-permeable liner in a spaced-apart relation to the body liquid-permeable liner.

13. The incontinence garment of claim 12, wherein a second front portion moisture barrier is interposed between the body liquid-permeable liner and the front portion liquid-permeable liner.

14. The incontinence garment of claim 1, wherein the body forms a length external angle of between about 70 degrees and about 100 degrees.

15. The incontinence garment of claim 1, wherein the body forms a width internal angle of between about 85 and about 105 degrees.

16. The incontinence garment of claim 1, wherein the ratio of a width of the body along a short axis to a length of the body along a long axis is between about 45 percent and about 65 percent.

17. A incontinence garment, comprising:
a chassis, the chassis defining an interior and an exterior, the chassis comprising:
a chassis moisture barrier;
a chassis absorbent, the chassis absorbent positioned inboard of the chassis moisture barrier;
a chassis liquid-permeable liner, the chassis liquid permeable liner positioned inboard of the chassis absorbent; and
an aperture, having a perimeter, the aperture defining an opening between the chassis interior and at least the chassis moisture barrier; and
a body, having a perimeter, the body perimeter positioned adjacent the aperture perimeter, the body comprising:
a shell, the shell forming a cavity, the cavity adapted to hold male genitalia;
a body absorbent, the body absorbent at least partially disposed within the cavity; and
a body liquid-permeable liner, the body liquid-permeable liner attached to the shell, a portion of the body liquid-permeable liner in a spaced-apart relation to the body absorbent, the body liquid-permeable liner and the body absorbent describing a gap and forming at least a partial chamber, wherein, while the incontinence garment is being worn, the body liquid-permeable liner is under sufficient tension to at least partially maintain the gap and to at least partially maintain the at least partial chamber and to hammock male genitalia within the cavity and away from the body absorbent; and, wherein:
the body forms a length external angle of between about 70 degrees and about 100 degrees;
the body forms a width internal angle of between about 85 degrees and about 105 degrees; and
the ratio of a width of the body along a short axis to a length of the body along a long axis is between about 45 percent and about 65 percent;
a first incontinence garment moisture barrier, the first incontinence garment moisture barrier interposed between the body absorbent and the chassis absorbent; and
a second incontinence garment moisture barrier, the second incontinence garment moisture barrier interposed between the body liquid-permeable liner and the chassis liquid-permeable liner.
